(19) United States
(12) Patent Application Publication
(10) Pub. No.: US 2018/0261322 A1
(43) Pub. Date: Sep. 13, 2018

(54) SMART SYRINGE: MONITORING MEDICAL INTERVENTION INFORMATION

(71) Applicant: KONINKLIJKE PHILPS N.V., EINDHOVEN (NL)

(72) Inventors: Limei CHENG, IRVINE, CA (US); Dong WANG, SCARSDALE, NY (US)

(21) Appl. No.: 15/761,160

(22) PCT Filed: Sep. 8, 2016
(86) PCT No.: PCT/IB2016/055340

§ 371 Date: Mar. 19, 2018

Related U.S. Application Data
(60) Provisional application No. 62/222,413, filed on Sep. 23, 2015.

(51) Int. Cl. G16H 40/63; A61B 5/00; A61M 5/20; A61M 5/142; A61M 5/172; A61M 5/168

(52) U.S. Cl. CPC G16H 40/63; H04L 67/10; A61M 5/20; A61M 5/142; A61M 5/1723; A61M 5/16804; A61M 2205/3584; A61M 2205/502; A61M 2205/70; A61M 2205/3334; A61M 2205/6081; A61M 2205/3368; A61M 2205/6072; A61M 2205/6054; A61M 2230/06; A61M 2230/30; A61M 2230/20; A61M 2205/505; A61M 2205/3324; A61M 2205/50; A61B 5/002

(57) ABSTRACT

The following relates to an improved system and method for delivering medication to a patient through a syringe. A intravascular (IV) device may include a syringe, a temperature sensor, flow detector, and a reader such as a barcode reader or radiofrequency ID (RFID) reader. The reader may measure information that indicates a medication type. Information measured by the IV device may be used along with patient vital sign information to form a closed-loop feedback system to control the IV device.

### BACKGROUND

The following relates to an improved system and method for delivering medication to a patient through a syringe.

Syringes, infusion pumps, and other intravascular (IV) medication delivery devices are an efficient and effective way to deliver medication to a patient. Medical institutions have been known to use various methods to improve use of syringes. For example, the traditional syringe is operated manually, but it is also known to automate operation of a syringe. In addition, medical institutions purchase syringes prefilled with medication in order to streamline the medication delivery process. Infusion pumps often employ electronic feedback control to deliver precise flow rates, which may be programmed in accord with a schedule, e.g. delivering an initial “bolus” at a high flow followed by a longer term but much lower flow rate thereafter. Infusion pumps also may incorporate sensors to monitor parameters such as flow rate, remaining fluid volume (and/or equivalent remaining infusion time), and various alerts and/or alarms to indicate events such as flow blockage. In some cases fluid temperature may also be a monitored parameter.

However, most medical information recorded in conjunction with IV delivery devices are recorded manually, which leads to limitations on the information being recorded. For example, the information recorded typically only includes starting time, ending time, and dosage. The starting and ending times are often approximate, for example recorded as the time of the nurse's visit to the patient's room during which the infusion was started this manually recorded time may differ by tens of seconds, or even minutes, when compared with the time of actual start of infusion. The end time may be only an approximate value computed from the (already approximate) start time and the expected total infusion time. Similar approximations may be entailed in estimating other events such as the end of the highflow rate initial bolus. This lack of time precision can be especially problematic when multiple medications are administered at about the same time, or when the infusion is started or stopped in conjunction with other events or other therapies.

The following provides a new and improved systems and methods which overcome the above-referenced problems and others.

### SUMMARY

In one aspect, a patient monitor includes: a wired or wireless communication interface configured to communicate with an intravascular (IV) device; a wired or wireless vital sign sensors interface; a display component; and an electronic device programmed to: receive measured medication information as a function of time from the IV device via the wired or wireless communication interface; receive vital sign information as a function of time from one or more vital sign sensors connected to the wired or wireless vital sign sensors interface; and simultaneously display on the display component time-synchronized representations of both the vital sign information as a function of time and the measured medication information as a function of time.

The measured medication information as a function of time may include flow rate as a function of time, and medication temperature as a function of time. The electronic device may be programmed to simultaneously display on the display component trend lines of both the vital sign information as a function of time and the measured medication information as a function of time with the time axes of the trend lines aligned horizontally or vertically and synchronized in time. The electronic device may be further programmed to change the color of the trend line of the measured medication information in accordance with a flow rate change. The electronic device may be programmed to simultaneously display on the display component a current numeric value or values of both the vital sign information and a current numeric value or values of the measured medication information as a function of time wherein all displayed current numeric values are updated in real-time. The wired or wireless communication interface may be configured to communicate with a plurality of IV devices and the electronic device is programmed to receive measured medication information as a function of time from a plurality of IV devices via the wired or wireless communication interface and to simultaneously display on the display component time-synchronized representations of all of the vital sign information as a function of time and the measured medication information from each IV device as a function of time. The electronic device may be further programmed to: determine a control signal for controlling the IV device based at least on the vital sign information as a function of time; and send the determined control signal to the IV device.

In another aspect, an intravascular (IV) device in communication with a data collection system includes: a flow controller configured to automatically control flow rate of a medication; a flow detector configured to measure flow rate of the medication; a wired or wireless communication interface configured to communicate with a patient monitor to receive patient vital sign data; and an electronic device programmed to: receive a vital sign signal via the wired or wireless communication interface; generate a flow rate control signal based on the vital sign signal and the measured flow rate; reset the flow rate to a default flow rate if the vital sign signal is lost. The reader may be a barcode reader or a radio frequency identification (RFID) reader. The intravascular device may be a syringe or may be an infusion pump. The intravascular device may further include: a temperature sensor housed within a syringe body; and a reader, connected by a cord to the syringe body, configured to identify a medication type.

In another aspect, a system includes: an intravascular (IV) device, comprising: a flow detector configured to measure a flow rate of a medication; a temperature sensor configured to measure a temperature of the medication; a reader, connected by a cord to the syringe body, configured to identify a medication type; an electronic device programmed to: receive a control signal from the patient monitor; set a flow rate based on the received control signal; a patient monitor, comprising: an electronic device programmed to: receive measured medication information from the IV device; receive vital sign information. The system may also include a display configured to display the vital sign information alongside the measured medication information.

The system may include at least two IV devices. The IV device may be programmed to reset the flow rate to a default flow rate if the control signal is lost. The electronic device of the IV device may be programmed to reset the flow rate to a default flow rate if the patient vital sign information is lost. The measured medication information comprises a flow rate measured by the flow detector and a temperature measured by the temperature sensor. The display may be further configured to display the vital sign information alongside the measured medication information in a time-synchronized manner. The display may be further configured to change the color of the waveform in accordance with a flow rate change. The displayed vital sign information may include electrocardiogram (EKG), arterial blood pressure, and SpO2 information displayed together in waveform format.

One advantage resides in a medication delivery system with a reduced probability of error in delivering medication.

Another advantage resides in a medication delivery system with improved data-recording capabilities.

Another advantage resides in earlier availability of patient data, resulting in earlier disease detection and diagnosis.

Another advantage resides in a graphic user interface that displays medication information together and time-synchronized with vitals, which facilitates analysis of correlations between medications and physiological changes.

Other advantages will become apparent to one of ordinary skill in the art upon reading and understanding this disclosure. It is to be understood that a specific embodiment may attain, none, one, two, more, or all of these advantages.

The invention may take form in various components and arrangements of components, and in various steps and arrangements of steps. The drawings are only for purposes of illustrating the preferred embodiments and are not to be construed as limiting the invention.

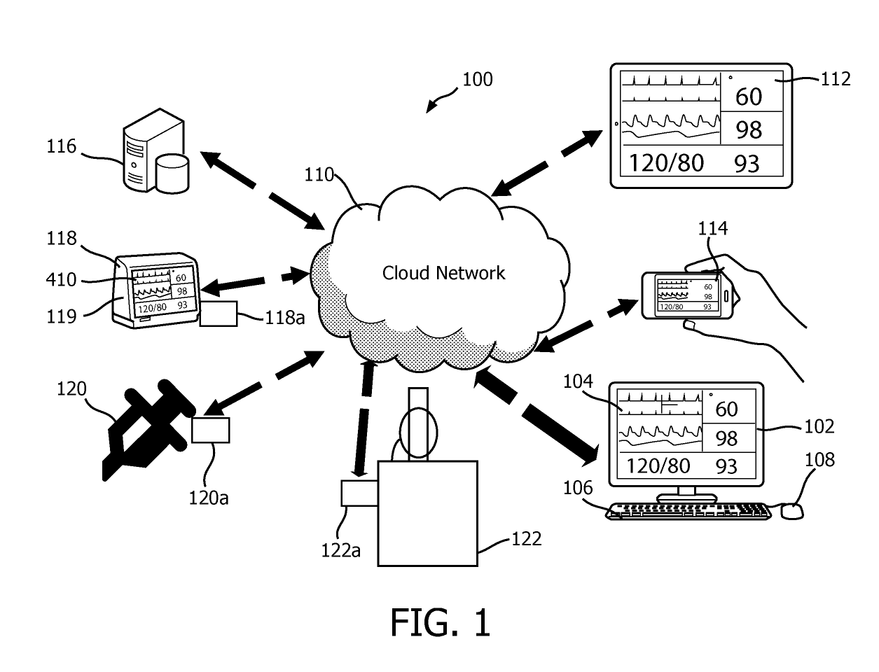

FIG. 1 diagrammatically shows a medical institution network.

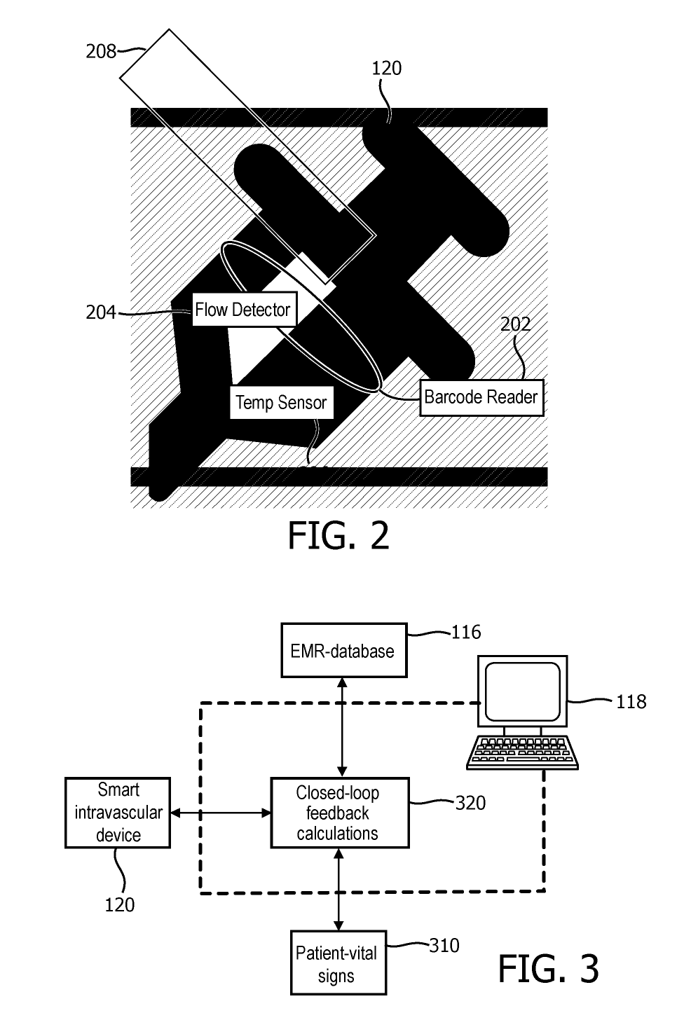

FIG. 2 is an exemplary illustration of a smart syringe.

FIG. 3 is a diagrammatical illustration of closed-loop feedback in accordance with an aspect of the present invention.

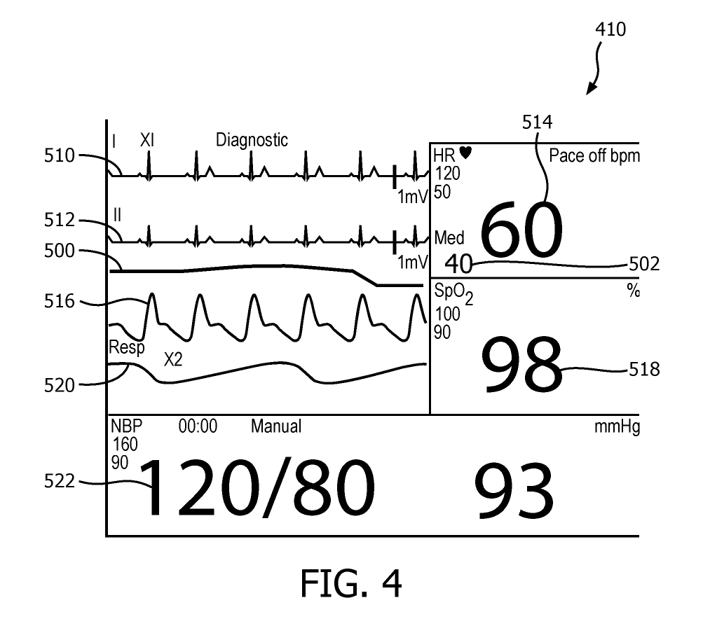

FIG. 4 illustrates a display in accordance with the systems and methods described herein.

### DETAILED DESCRIPTION

The following relates to a “syringe” which may more generally encompass any intravascular (IV) medication delivery device, e.g. a conventional plunger-based syringe with a hypodermic needle, or an infusion pump-based IV medication delivery device with a hypodermic needle or catheter for vascular entry. The IV device may include “smart” hardware such as a medication identification tag (e.g. RFID or barcode), flow meter, and/or a medication temperature sensor.

FIG. 1 shows a medical institution network 100, which the smart IV device 120 and/or infusion pump 122 or other IV medication delivery device may be integrated into. With reference thereto, a computer 102 includes a display 104 and illustrative keyboard 106. More generally, the computer 102 may include various user input devices such as a keyboard 106, a mouse 108, trackball or other pointing device, a touchscreen display component of the display 104, or so forth. Cloud network 110 may connect to the computer 102, as well as connect to other devices including tablet 112 and smartphone 114. In this way the devices 102, 112 and 114 may communicate with each other, as well as communicate with electronic medical record (EMR) database 116, patient monitor 118, smart syringe 120, and infusion pump 122. Any device connected to the cloud network 110 can communicate with any other device connected to the cloud network 110 though the cloud network. Alternatively, communication between devices may be regulated or only certain devices may communicate with certain other devices via the network 110. Further, while a cloud network is shown, other communication networks may be employed such as a wireless (e.g. WiFi) hospital network, a wired hospital Ethernet, the Internet (preferably using secure connections), various combinations thereof, or so forth. Multiple IV medication delivery devices such as the illustrative smart syringe 120 and the infusion pump 122 may be connected to the network 110; and multiples of any other individual component may also be connected to the network 110. To enable communication, patient monitor 118 includes communication unit 118a; smart syringe 120 includes communication unit 120a; and infusion pump 122 includes communication unit 122a, and the various other devices 102, 112, 114 each include communication units (e.g. the built-in 4G and/or WiFi communication units of the tablet 112 and cellphone 114).

One improvement of the approaches described herein is the integration of medication information as a function of time from such a “smart” IV medication delivery device with an overall patient monitoring paradigm. To this end, the IV device 120, 122 further includes a wired (e.g. USB) or wireless (e.g. Bluetooth) link to port medication information (e.g. type, flow rate, temperature) to a data collection system that also collects vital signs. In some embodiments, this link is via the WiFi, Ethernet, cloud, or other network 110. The medication information is time stamped (either at the IV device using an on-board clock or at receipt in the data collection system) and thus can be correlated in real time with measured vital signs, anesthesia delivery, or so forth.

FIG. 2 illustrates the exemplary smart syringe 120 of FIG. 1 in greater detail. The smart syringe 120 includes barcode reader 202, temperature sensor 206, flow detector 204, and flow controller 208. These components enable: measurement of dosing information (including flow rate during a continuous flow administration) during intravenous intervention without disturbing the drug administration; recordation of medication information; and transmission of the medication data to the patient monitor for display and storage.

In more detail and with continuing reference to FIG. 2, the barcode reader 202 identifies drug information including drug name, lot number and expiration date, etc. The barcode reader 202 may be located outside of the smart syringe 120 and may be connected with a cord. Alternatively, a radio frequency identification (RFID) reader or near-field communication (NFC) receiver or magnetic strip reader or the like can be integrated in the smart syringe 120 to read the drug information that is stored in an RFID tag, magnetic strip or so forth attached to or embedded in the container holding the administered drug.

The flow detector 204 measures the flow rate continuously in real-time. Measurements may be taken for both bolus injections and continuous inputs. A sensor of the flow detector 204 can be deployed at the entrance of the syringe close to its bottom inside of syringe where the medication flows into the syringe needle.

Temperature sensor 206 detects the temperature of drug. If the temperature is outside of a certain desired range, a warning can be displayed on the patient monitor. A detector of the temperature sensor 206 can be placed close to its bottom inside of smart syringe 120.

These are illustrative data collection components, and a given “smart” syringe may not include all sensors 202, 204, 206, and/or may include other sensors such as (by way of non-limiting illustration) a pH sensor detecting medication pH, a pressure sensor, or so forth. In addition, in the example of FIG. 2, the illustrated smart syringe 120 is in the general syringe format which has a jig to fit different size and multiple usages.

In like fashion, the infusion pump 122 includes sensors to monitor the medication infusion delivery performed by the infusion pump 122. As previously noted, infusion pumps are generally programmed to deliver medication intravascularly at a precise flow rate, which may be in accord with a schedule, e.g. delivering an initial high flow rate bolus for a short initial time interval followed by continuous flow at a much lower rate. Infusion pumps typically include flow rate sensors for this control purpose, and may compute other useful information such as remaining fluid volume and/or equivalent remaining infusion time. The infusion pump 122 is typically also programmed to provide various alerts and/or alarms to indicate events such as flow blockage. It is also contemplated to include a temperature sensor analogous to the temperature sensor 206 of the syringe 120.

The medication information acquired by such sensors (e.g. the medication identification sensor 202, the flow detector 204, temperature sensor 206 of the syringe 120, or similar sensors of the infusion pump 122) are communicated via the wired or wireless communication interface 120a, 122a to the patient monitor 118 via its wired or wireless communication interface 118a. Except for the illustrative medication identification sensor 202, these sensors 204, 206 provide medication information as a function of time. The IV medication delivery device 120, 122 includes suitable electronics (e.g. a microprocessor or microcontroller and ancillary components such as electronic memory, features not illustrated) to format the data from these sensors 202, 204, 206 into a data stream of samples. For example, in one approach, the data stream includes an initial header containing the medication identification provided by the sensor 202 and perhaps other information such as settings of the IV medication delivery device, followed by flow rate and temperature samples (or more generally, measured medication information samples) as a function of time. The data stream is communicated to the patient monitor 118 via the communication interfaces 120a, 118a (for the syringe 120) or via the communication interfaces 122a, 118a (for the infusion pump 122). Each sample is time stamped at the IV medication delivery device 120, 122 and/or at receipt at the patient monitor 118. The patient monitor can then display the simultaneously display on a display component 410 (see FIG. 4) time-synchronized representations of both vital sign information as a function of time received at the patient monitor 118 via a wired or wireless vital sign sensors interface 119 (see FIG. 1) and the measured medication information as a function of time. In this way, the nurse, doctor or other medical profession can readily observe the impact of the medication on the patient's vital signs and correlate medications with changes in the patient.

Another contemplated application is to provide closed-loop control of IV medication delivery based on real-time vital signs. For example, a medication flow rate can be increased or decreased in correlation with change in heart rate, respiration rate, or other vital signs. For FDA (in the United States, or other governmental regulatory) approval, closed-loop control would preferably employ well-defined rules and optionally includes hard limits on the minimum and/or maximum flow rate. These limits may be prescribed by the physician as part of the medication order. Closed-loop control of other medical devices based on the real-time medication information is also contemplated, e.g. an anesthesia machine could be controlled in part based on medication information (e.g. interlocked to ensure appropriate medications are administered in conjunction with the anesthesia). The closed-loop control could be implemented on the smart IV medication delivery device 120 by transmitting vital sign data from the patient monitor 118 to the IV medication delivery device 120, 122, or can be implemented on a patient monitor 118 by having the patient monitor compute the flow rate based on the vital signs and send an appropriate flow control signal to the IV medication delivery device 120, 122. The smart IV device 120, 122 could be programmed to go to a default flow rate if the vital sign data or flow control signal from the patient monitor 118 is lost.

The real-time vital signs may be stored along with real-time medication information in EMR 116. Advantageously, the real-time vital signs and real-time medication information may be stored as a function of time. Further advantageously, the real-time vital signs may be stored in a time-synchronized manner with the real-time medication information. One way to time-synchronize the data is to use a clock of the patient monitor 118 to time stamp the patient vital sign data and medication information when they are received at the patient monitor 118. Another way to time-synchronize the data is to use a clock on the smart IV device 120 to time stamp the medication information, and to use the clock on the patient monitor 118 to time stamp the patient vital sign data; in this example, the delay from transmission between the smart IV device 120 and patient monitor 118 could be adjusted for, or accepted as an acceptable amount of error if the transmission delay is deemed to be negligible.

FIG. 3 shows an embodiment where closed-loop control is implemented at the patient monitor 118. With reference thereto, smart IV device 120 (e.g. a smart syringe), EMR database 116, and patient vital sign data all are sent to patient monitor 118. Patient monitor 118 performs closed-loop feedback calculations 320, which may be used to control smart IV device 120. Also, the data is transferred through a wired (e.g. USB) or wireless (e.g. Bluetooth) link into the patient monitor 118. Wireless data transfer is an upgrade for the smart syringe 120 and is an optional feature.

With reference to FIG. 4, all data (vital signs and medication information) can be displayed in real time on a graphical display 410 of a single patient monitor 118, which facilitates a holistic interpretation of patient vital signs as influenced by the administered medications. This is especially useful where the patient may be receiving a number of different medications at the same time, with various medications being interrupted (for example to replace the medication fluid bag) and/or administered in bolus form over short time intervals. Thus, for example, the effect of a bolus on heart rate can be immediately observed via two parallel trend lines, one plotting medication flow rate and the other plotting heart rate. Patient monitor 118 is often located bedside a patient.

The illustrative display of FIG. 4 which may be suitably shown on the graphical display 410 of the patient monitor 118 can also be applied to a web-based applications shown on the display component on any computer 102, tablet 112 or smart phone 114 or so forth. In one example, this allows remote access of the same ventilation history information of the patient for a physician to make a clinical decision such as weaning or adjust ventilator setting in a more effective and more convenient way. Advantageously, this allows for earlier disease detection and diagnosis.

The display of illustrative FIG. 4 shows the medication data (e.g. flow rate) collected by smart syringe 120 as a trend line 500 and/or as a current numeric value 502 that is updated in real-time. The representation(s) 500, 502 as a function of time of the medication information is/are simultaneously displayed on the display component 410 time-synchronized with representations of vital sign information as a function of time, such as illustrative electrocardiogram (EKG) lead traces 510, 512 (i.e., trend lines of the EKG lead signals), a heart rate displayed as a current numerical value 514 that is updated in real-time, a plethysmograph signal trend line 516 and derived SpO2 signal displayed as a current numerical value 518 that is updated in real time, and a respiration signal displayed as a trend line 520. It is to be appreciated that “real time” in this context refers to the values being updated as new samples arrive, which may occur at different time intervals depending upon the sampling rate, but is typically on the order of one sample per minute or faster. Some vital sign data may be updated less frequently and hence might not be deemed to be “real time” values for example, blood pressure data displayed as current values 522 may be updated only every few minutes or so if the blood pressure is measured using an arm cuff that is inflated at intervals of every few minutes. Also, if multiple smart syringes are used for several medications, all drug data together with the vitals may be displayed in waveform format or numeric format. For each waveform of the medication, the color of the waveform can be changed in accordance with the flow rate change. The data are preferably also labeled on the display. In the illustrative example, the generic label “Med” is used as the label for the medication trend line 500, but in other embodiments this generic label is replaced by the medication name, which may be manually entered into the patient monitor 118 or, in more automated embodiments, may be received from the IV medication delivery device 120, 122, for example having been read by the barcode reader 202 and transmitted to the patient monitor 118 in a header of the medication data stream.

Advantageously, the medical injection intervention information 500, 502 is displayed together with relevant patient vital sign signals 510, 512, 514, 516, 520, 522 in a same window. The IV medication delivery device 120, 122 can identify the drug information, detect dosing information such as drug flow rate in real-time. Thus, the IV medication information and patient vital sign data may be synchronized in the time axis to help clinicians better assess patient status and determine whether a medical intervention is effective. For example, the illustrative trend lines 500, 510, 512, 516, 520 preferably have the same time axis, that is, the trend lines are aligned horizontally (time is along the horizontal axis in the trend lines of FIG. 4) and synchronized in time. In an alternative embodiment, the trend lines may employ vertical time axis, again preferably aligned (vertically in this case) to provide the time synchronization. In the case of the numeric values 502, 514, 518, these are time synchronized in that each numeric value 502, 514, 518 displays the current value of the vital sign or medication data, so they are time-synchronized at the current time. (Again, due to differences in sampling rate, the update latency of these current values may be different for the different numerical representations, but they are time-synchronized within the latency intervals imposed by the sampling rates).

It will be further appreciated that the techniques disclosed herein may be embodied by a non-transitory storage medium storing instructions readable and executable by an electronic data processing device (such as the computer 102, patient monitor 118, tablet 112, or smartphone 114) to perform the disclosed techniques. Such a non-transitory storage medium may comprise a hard drive or other magnetic storage medium, an optical disk or other optical storage medium, a cloud-based storage medium such as a RAID disk array, flash memory or other non-volatile electronic storage medium, or so forth.

Of course, modifications and alterations will occur to others upon reading and understanding the preceding description. It is intended that the invention be construed as including all such modifications and alterations insofar as they come within the scope of the appended claims or the equivalents thereof.

1. A patient monitor comprising:
a wired or wireless communication interface configured to communicate with an intravascular (IV) device;
a wired or wireless vital sign sensors interface;
a display component; and
an electronic device programmed to:
receive measured medication information as a function of time from the IV device via the wired or wireless communication interface;
receive vital sign information as a function of time from one or more vital sign sensors connected to the wired or wireless vital sign sensors interface;
simultaneously display on the display component time-synchronized representations of both the vital sign information as a function of time and the measured medication information as a function of time; and
change the color of the trend line of the measured medication information in accordance with a flow rate change.

2. The patient monitor according to claim 1, wherein the measured medication information as a function of time comprises flow rate as a function of time, and medication temperature as a function of time.

3. (canceled)

4. (canceled)

5. The patient monitor according to claim 1, wherein the electronic device is programmed to simultaneously display on the display component a current numeric value or values of both the vital sign information and a current numeric value or values of the measured medication information as a function of time wherein all displayed current numeric values are updated in real-time.

6. The patient monitor according to claim 1, wherein the wired or wireless communication interface is configured to communicate with a plurality of IV devices and the electronic device is programmed to receive measured medication information as a function of time from a plurality of IV devices via the wired or wireless communication interface and to simultaneously display on the display component time-synchronized representations of all of the vital sign information as a function of time and the measured medication information from each IV device as a function of time.

7. The patient monitor according to claim 1, wherein the electronic device is further programmed to:
determine a control signal for controlling the IV device based at least on the vital sign information as a function of time; and
send the determined control signal to the IV device.

8. An intravascular (IV) device in communication with a data collection system, the device comprising:
a flow controller configured to automatically control flow rate of a medication;
a flow detector configured to measure flow rate of the medication;
a wired or wireless communication interface configured to communicate with a patient monitor to receive patient vital sign data; and
an electronic device programmed to:
receive a vital sign signal via the wired or wireless communication interface;
generate a flow rate control signal based on the vital sign signal and the measured flow rate; and
reset the flow rate to a default flow rate if the vital sign signal is lost.

9. The intravascular (IV) device of claim 8, wherein the reader is a barcode reader or a radio frequency identification (RFID) reader.

10. (canceled)

11. The intravascular (IV) device of claim 8, further comprising:
a temperature sensor housed within a syringe body; and
a reader, connected by a cord to the syringe body, configured to identify a medication type.

12. A system including:
an intravascular (IV) device, comprising:
a flow detector configured to measure a flow rate of a medication;
a temperature sensor configured to measure a temperature of the medication;
a reader, connected by a cord to the syringe body, configured to identify a medication type;
an electronic device programmed to:
receive a control signal from a patient monitor;
set a flow rate based on the received control signal;
timestamp measured medication information;
the patient monitor, comprising:
an electronic device programmed to:
receive measured the medication information from the IV device;
receive vital sign information;
timestamp the vital sign information; and
adjust the control signal for a delay in transmission between the IV device and the patient monitor; and
a display configured to display the vital sign information alongside the measured medication information.

13. The system of claim 12 including at least two IV devices.

14. The system of claim 12, the electronic device of the IV device programmed to reset the flow rate to a default flow rate if the control signal is lost.

15. The system of claim 12, the electronic device of the IV device programmed to reset the flow rate to a default flow rate if the patient vital sign information is lost.

16. (canceled)

17. The system of claim 12, wherein the measured medication information consists of a flow rate measured by the flow detector and a temperature measured by the temperature sensor.

18. The system of claim 12, wherein the display is further configured to display the vital sign information alongside the measured medication information in a time-synchronized manner.

19. The system of claim 12 wherein the display is further configured to change the color of the waveform in accordance with a flow rate change.

20. The system of claim 12, wherein the displayed vital sign information includes electrocardiogram (EKG), arterial blood pressure, and SpO2 information displayed together in waveform format.
